(19) United States
(12) Patent Application Publication
(10) Pub. No.: US 2022/0378342 A1
(43) Pub. Date: Dec. 1, 2022

(54) WEARABLE AUTONOMOUS BIOMIMETIC SWEAT SENSOR FOR PRECISION NUTRITION

(71) Applicant: California Institute of Technology, Pasadena, CA (US)

(72) Inventors: Wei Gao, Pasadena, CA (US); Minqiang Wang, Pasadena, CA (US); Yiran Yang, Pasadena, CA (US); Jihong Min, Pasadena, CA (US)

(73) Assignee: California Institute of Technology, Pasadena, CA (US)

(21) Appl. No.: 17/824,798

(22) Filed: May 25, 2022

Related U.S. Application Data
(60) Provisional application No. 63/192,968, filed on May 25, 2021.

(51) Int. Cl. A61B 5/145; A61B 5/00; A61B 5/1477

(52) U.S. Cl. CPC A61B 5/14546; A61B 5/681; A61B 5/1477; A61B 5/14521; A61B 5/0002; A61B 5/14532; A61B 5/4866

(57) ABSTRACT

Systems and methods for a microfluidic biosensor patch and health monitoring system may include an iontophoresis module, a multi-inlet microfluidic sweat collection and sampling module, and a molecularly imprinted polymer (MIP) organic compound sensor module. An iontophoresis module may provide for stimulation of a biofluid sample. A biofluid may be a sweat sample. Stimulation may be achieved via electrostimulation and/or application of hydrogel. A microfluidic sweat collection and sample module may include several adhesive layers with carefully designed inlets, channels, a reservoir, and an outlet for the efficiently collection and sampling of biofluid. A MIP sensor module may quickly and accurately identify concentrations of key metabolites present in a biofluid sample which may indicate certain health conditions.

### CROSS-REFERENCE TO RELATED APPLICATIONS

This application claims the benefit of U.S. Provisional Patent Application No. 63/192,968, filed May 25, 2021, the content of which is incorporated herein by reference in its entirety.

### GOVERNMENT FUNDING DISCLAIMER

This invention was made with government support under Grant No. NR018271 awarded by the National Institutes of Health. The government has certain rights in the invention.

### TECHNICAL FIELD

The present disclosure relates generally to systems and methods for biomarker monitoring. In particular, some implementations may relate to systems and methods for wearable biosensor monitoring of key metabolites using human sweat samples.

### BACKGROUND

Wearable bioelectronic technology offers many advantages for personalized health monitoring. Wearable devices are non-invasive and present less user error than other monitoring methods. Additionally, wearable devices offer the potential to monitor health status over time as opposed to collecting a sample that reflects health status at only a snap shot in time. This type of real-time monitoring offers more accurate and individualized diagnosis, treatment, and prevention for health conditions. Specifically wearable devices can measure pulse, respiration rate, temperature and other health status indicators.

Sweat sensors are one type of wearable bioelectronic sensors that are particularly desirable because sweat contains many key biomarkers including electrolytes, metabolites, amino acids, hormones, and drug levels. However, existing sweat sensors face several key problems. First, existing sensors lack an effective continuous monitoring strategy. They employ only ion-selective and enzymatic electrodes and/or direct oxidation of electroactive molecules. Therefore, these sensors are only able to measure a limited set of biomarkers such as electrolytes, glucose, and lactate. These biomarkers alone do not provide a full enough picture of a human subject's health status to serve as an effective preventative tool. Additionally, these sensors often require a large sample of sweat to provide accurate analysis of biomarkers. This requires a larger and more powerful device which may not be suitable as a wearable. Therefore, monitoring and especially continuous monitoring presents a challenge due to these high power needs and the need for power storage. Existing models present additional challenges including that they require complex fabrication, are difficult to reproduce in large quantities in an affordable way, and are fragile, making them not suitable as wearable devices for long periods.

For at least these reasons, the current “gold standard” for measuring metabolites and other key biomarkers in the body is blood testing. Blood testing has several drawbacks including that it is invasive, as it requires withdrawal of blood from the veins. Also, accurate blood testing and/or blood testing needing larger samples generally requires a human subject to come to the lab and be tested. Because of the lab requirement and invasiveness, blood testing is generally only performed at a snapshot in time. This means that in many cases, unless a patient is experiencing a flare or other type of health episode at the time of testing, the testing many not reveal any unusual metabolite levels until a health problem has become severe. Additionally, this episodic testing makes it extremely difficult to determine what factors may influence a patient's change in metabolite levels over time. Further, blood testing is very delicate as samples can be easily compromised by oxidation and other factors. Therefore, expensive equipment is required to process each sample, resulting in lengthy processing times and less testing overall.

Because of the lack of effective continuous monitoring strategies and high power needs, currently existing wearable health monitoring systems are unable to measure key biomarkers in a comparable way to blood testing. An effective wearable system would be highly desirable as blood testing is invasive, expensive, and offers limited health information over time.

### SUMMARY

Systems and methods are described herein related to wearable biosensors capable of continuous health monitoring. Health monitoring may include monitoring of key metabolites and may support precision nutrition and/or personalized medicine. Such a system may leverage several strategies including integration of laser-engraved graphene, redox-active nanoreporters, biomimetic “artificial antibodies,’ and in situ regeneration technologies to offer several advantages. These advantages may allow for more precise monitoring over lengthier periods of time and may enable more sensitive health monitoring. For example, the systems and methods disclosed herein may offer monitoring and analysis of trace-level metabolites and nutrients including all essential amino acids and vitamins. Other health conditions such as fatigue and infection, including viral infection, may be monitored.

Such a system may also leverage localized sweat simulation, microfluidic sweat sampling, and on-board signal calibration to offer additional advantages. These additional advantages may include prolonged monitoring which may continue both during states of exercise and states of rest. These additional advantages may also support a low-powered, light-weight and low-cost wearable device which can be easily reproduced and fabricated, leading to more accessibility and greater sample sizes for machine learning operation. Advantages may also support a device that is comfortable, non-invasive, and easily worn by a human patient as needed, including for extended periods of time.

Amino acids are organic compounds that are present in the human body and in food sources. Concentrations of amino acids may vary depending on many factors including dietary intake, genetic predisposition, gut microbiota, environmental factors, lifestyle factors including sleep and exercise, and other factors. The concentrations of amino acids present in human bodily fluids, including sweat and blood, can provide important information about the health of an individual. For example, elevated levels of branched-chain amino acids (BCAAs) including for example, leucine (Leu), isoleucine (Ile), and Valine (Val) may be correlated with certain health conditions including obesity, insulin resistance, diabetes, cardiovascular disease, and pancreatic cancer. Deficiencies in amino acids, including, for example, arginine and cysteine, may indicate immune suppression and/or reduced immune-cell activation. Imbalances with other compounds, such as Tryptophan (Trp), tyrosine (Tyr) and phenylalanine (Phe), which are needed to support neurotransmitters such as serotonin, dopamine, norepinephrine, and epinephrine, may indicate neurological and/or mental health conditions. Other metabolic indicators involving, for example, Leu, Phe, and vitamin D, may be linked with severity, vulnerability, and mortality related to viral infections including COVID-19.

Wearable sensors integrated with telemedicine could support safe and efficient monitoring of individual health which would allow for timely intervention for viral infection, including COVID-19, both for an individual and for communities.

A universal wearable biosensing strategy may combine mass-produced laser-engraved graphene (LEG), electrochemically synthesized redox-active nanoreporters (RARs), biomimetic molecularly imprinted polymer (MIP)-based ‘artificial antibodies’, in situ regeneration and calibration technologies. Such a strategy may allow for sensitive, selective, and continuous monitoring of wide range of trace-level biomarkers in biofluids including all nine essential AAs, and essential vitamins. Such a strategy may integrate seamlessly with prolonged iontophoresis-based on-demand sweat induction, efficient microfluidic-based sweat sampling, and in situ signal processing and wireless communication to get an autonomous health platform.

A sensor patch may be flexible and disposable and may have includes iontophoresis electrodes, a microfluidic module, an analyte sensor module, a temperature sensor, and an electrolyte sensor. In an embodiment, an analyte sensor module may be a multiplexed MIP nutrient sensor array. A sensor and its electrodes may be designed using LEG technology. LEG fabrication may enable large scale production of biosensor systems, via CO₂ laser engraving, at relatively low cost. A sensor patch may also include a miniaturized module with iontophoresis control, in situ signal processing and wireless communication via Bluetooth. A sensor patch may be integrated with a mobile application for displaying, processing, and storing collected health data. A sensor patch may also be integrated into a smart watch.

Sweat may be a desirable biofluid to measure because sweat is rich in metabolites and builds up on/near the surface of skin. This makes sweat comparatively inexpensive and noninvasive to harvest and analyze versus other biofluids like blood. However, sweat composition varies highly on an individual basis and requires sensitive technology for accurate measurements. One approach may be to measure the different between two oxidation peak heights. For example, a small peak may be measured before a target molecule is bound to a binding site embedded in the LEG sensor. Then the difference between the small peak and a substantially higher peak measured after recognition and binding of a target molecule in an MIP template in an LEG sensor may be measured. A sensor may also be calibrated to account for temperature effects on sensitivity in real time. For instance, a reading from LEG-based-strain-resistive temperature sensor and ion selective Na⁺ sensor may be taken. These techniques may support accurate continuous on-body monitoring of sweat for metabolites.

Application of hydrogel may be used for non-invasive and non-painful sweat induction. For example, carbachol/carbagel may be administered. This may offer long-term induction of sweat despite one time application of a small amount of carbagel. Sweat may then be collected in a microfluidic module. Such a module may be designed for optimal harvesting of sweat. Sweat may be repeatedly induced and sampled. The geometric design and features of the module may be selected for optimal sample efficiency. Key features may include the geometric design of the module, the number of inlets, the angle span between inlets, the orientation of inlet channels, and the flow direction into the reservoir.

Careful design of a selective binding MIP layer on an LEG may allow for sensitive and selective detection of AAs. MIPs are chemically synthesized biomimetic receptors formed by polymerizing functional monomers with template molecules. Here, a functional monomer, which may be, for example, pyrrole, and a crosslinker, which may be, for example, 3-Aminophenylboronic acid, may form a complex with a target molecule. Then, after polymerization, the functional groups of the functional monomer and crosslinker may be embedded in the polymeric structure on the LEG. Then, extraction of the target molecules may reveal binding sites on the LEG-MIP electrode that are complementary in size, shape, and charge to the target molecule. This may allow for detection without washing steps. Two detection strategies may be possible, including direct and indirect detection.

In an example embodiment, a target molecule may be detected directly. The oxidation of the target molecule in the MIP template may be able to be measured directly by differential pulse voltammetry (DPV). In DPV, the peak current height correlates directly to the analyte concentration. The direct approach may be effective for electroactive molecules. However, different electroactive molecules may be oxidized at similar potentials. Still, the approach is sufficiently selective and sensitive to distinguish between molecules. Several factors may influence sensitivity including the selected monomer, the selected crosslinker, the template ratios, the incubation periods, and other factors.

In another example embodiment, a target molecule may be detected indirectly. A RAR layer may be placed between the LEG and MIP layers. This configuration may enable rapid quantification. Target molecules may be selectively absorbed onto an imprinted polymeric layer which may decrease exposure of the RAR to the sample. Controlled-potential voltametric techniques such as DPV or linear sweeping voltammetry may be applied to measure the RAR's oxidization peak or reduction peak. A decrease in peak current height may correspond to an increase in the level of a particular analyte. In embodiment, Prussian Blue nanoparticles (PBNPs) may make up the RAR. An indirect approach may be effective for detecting the levels of non-electroactive metabolites.

In another embodiment, methods and systems may leverage a multi-template MIP to detect levels of many different metabolites through a single sensor. In many cases, measurements for several different key metabolites are needed to form a complete health picture. For example, amino acids, vitamins, minerals, and other metabolites including glucose and uric acid, may all be desired measurements.

Other features and aspects of the disclosure will become apparent from the following detailed description, taken in conjunction with the accompanying drawings, which illustrate, by way of example, the features in accordance with various embodiments. The summary is not intended to limit the scope of the invention, which is defined solely by the claims attached hereto.

### BRIEF DESCRIPTION OF THE DRAWINGS

The technology disclosed herein, in accordance with one or more various embodiments, is described in detail with reference to the following figures. The drawings are provided for purposes of illustration only and merely depict typical or example embodiments of the disclosed technology. These drawings are provided to facilitate the reader's understanding of the disclosed technology and shall not be considered limiting of the breadth, scope, or applicability thereof. It should be noted that for clarity and ease of illustration these drawings are not necessarily made to scale.

FIG. 1 is an example of a diagram showing a wearable sweat sensor.

FIG. 2 is an example of a diagram showing an exploded diagram showing a wearable sweat sensor patch.

FIG. 3A is an example of a diagram of a health monitoring system.

FIG. 3B is an example of a diagram of a health monitoring system.

FIG. 4 is an example of a flow diagram showing an iontophoresis method

FIG. 5 is an example of a flow diagram showing a preparation process of an LEG-MIP amino acid sensor.

FIG. 6 is an example of a flow diagram showing detection methods of an LEG-MIP amino acid sensor.

FIG. 7 is an example diagram of a microfluidic biofluid collection patch.

FIG. 8 is an example of a diagram of a microfluidic biofluid collection patch.

The figures are not intended to be exhaustive or to limit the invention to the precise form disclosed. It should be understood that the invention can be practiced with modification and alteration, and that the disclosed technology be limited only by the claims and the equivalents thereof.

### DETAILED DESCRIPTION

Wearable devices may offer highly desirable, non-invasive, and continuous monitoring of key health indicators. One type of desirable wearable is a sweat sensor. A carefully designed sweat sensor is particularly desirable because it may allow continuous on body monitoring of key health indicators. This kind of continuous analysis may allow for personalized medical care and nutrition for an individual based on that individual's particular balance of detected metabolites. Key metabolites may include essential amino acids and vitamins. Applications for a wearable sweat sensor may include dietary nutrition intake monitoring, evaluation of stress and central fatigue, evaluation for risk of metabolic syndrome, and evaluation for risk of severe viral infection, including COVID-19.

A laser-engraved graphene (LEG) sensor may be advantageous because it may be printed using a modified conventional printer. Printable wearable sensor patches may be fabricated on a large scale at a relatively low cost. This may allow for disposable sensor patches which may be worn by an individual for an extended of time, for instance twelve to twenty-four hours, and which may be replaced on a daily level. Low cost printable, wearable, and disposable patches offer the opportunity to replace a patch daily on a human subject and collect health information over a period of several days or weeks without invasive testing and the need for a human patient to come in to a physical laboratory for repeated testing. Monitoring may occur both during periods of exercise and at rest.

Continuous Sweat Induction and Collection

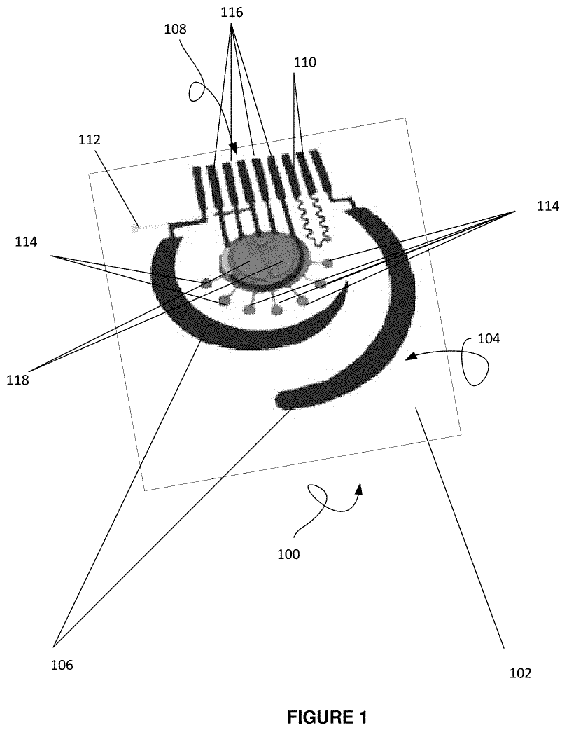

Referring now to FIG. 1 is an example of a sweat sensor patch 100. The sweat sensor patch may include a backing layer 102. The backing layer may be made of a polyimide film. The backing layer may also be made of some other material. A material with lightweight, heat and chemical resistant, and flexible material may be desirable. The backing layer may also include adhesive on the top surface (not shown in FIG. 1). The adhesive may be used to attach the sensor patch directly to the skin of a human subject. The sensor patch may include a biosensor array 104. The sensors may be patterned on the backing layer 102. Another layer, on top of the backing layer 102 may contain microfluidics. Microfluidics may include, for example, inlets 114, channels, and/or a reservoir. The additional layer may also contain adhesive on its top surface for attachment onto a skin surface. The biosensor array 104 may include several components including electrodes 106, biosensors 108, T sensors 110, and an outlet 112. The outlet 112 and inlets 114 may be patterned on the layer on top of the backing layer 102. The biosensor array 104 may be fabricated and printed onto the backing layer 102 using laser-engraved graphene (LEG) technology.

The electrodes 106 may provide a brief electrostimulation to the sweat glands of a human subject in a particular skin area. The electrostimulation may trigger the flow of sweat stimulating agents into the skin. A hydrogel agent (not shown in FIG. 1) may also be added to the sweat sensor patch 100. The hydrogel agent may be added in the same area as the electrodes 106. When the hydrogel agent comes into contact with a sweat gland, via the sweat sensor patch, the hydrogel agent may continue to stimulate the production of sweat.

The sweat sensor patch 100 may also include biosensors 108. Biosensors 108 may be configured to detect a wide variety of inorganic and organic compounds present in a biofluid sample. For example, metabolites, amino acids, vitamins, minerals, hormones, antibodies, and other compounds may be detected. A biosensor 108 may be a sodium sensor. A biosensor 108 may also be a glucose sensor. A sweat sensor patch 100 may also include a T sensor 110. The T sensor 110 may be a temperature sensor. A temperature reading, in conjunction with detected concentrations of key organic compounds, may provide an indication of health status. Additionally, a temperature measurement over time, along with correlated measurements of concentrations of key organic compounds, may provide indication about changing health status or may reveal fluctuations indicative of a disease or other health condition that would not be revealed by a one-time test, such as a blood test. An electrolyte reading (from sodium sensor, for example) may indicate a patient's hydration status and/or electrolyte balance. As with a temperature measurement, an electrolyte measurement, especially over a continuous period and in conjunction with other measurements, may reveal changing health status or fluctuations indicative of disease or a particular health condition.

The sweat sensor patch 100 may also include an outlet 112. The outlet 112 allows for the outflow of a collected sweat sample. The outlet 112 is configured such that the outflowing sweat sample does not interference with an incoming sweat sample. The sweat sensor patch 100 is configured to allow for collection and sample of refreshed sweat samples over an extended period of time. For example, the combination of electrode stimulation and hydrogel stimulation may induce a flow of sweat for a period of about 2 to 24 hours. The sweat sensor patch 100 may also include inlets 114 (not shown directly in FIG. 1 as the inlets 114 are on the layer above the backing layer 102). Incoming sweat samples may flow through the inlets 114 and then be directed into a reservoir for collection. (The reservoir is not shown directly in FIG. 1 as the reservoir is situated above the molecularly imprinted polymer (MIP) organic compound detection module). Once the sweat sample is analyzed by the biosensors 108 and/or MIP organic compound detection module 116, the sweat sample may flow out through the outlet 112, allowing for a refreshed sample to fill the reservoir and be analyzed.

A sweat sensor patch 100 may also include a MIP organic compound detection module. The MIP module may include a plurality of electrodes 116. MIP molecules may be added to two middle electrodes 118. The MIP module may comprise a layer on top of the LEG layer and may be carefully designed to achieve selective binding to identify the amounts of organic compounds and/or target molecules present in a collected sweat sample. The MIP may include a functional monomer and a crosslinker. The functional monomer may be, for example, pyrrole. The crosslinker may be, for example, 3-Aminophenylboronic acid. The functional monomer and the crosslinker may form a complex with a target molecule. After polymerization, the functional groups formed by the monomer, crosslinker, and target molecule may be embedded in the LEG. The target molecule can then be extracted such that the LEG has a binding site perfectly corresponding to the target molecule in size, shape, and charge, and in this way can detect the target molecule in the future.

In an embodiment, a sweat sensor patch 100 may include a miniature iontophoresis control module. The iontophoresis control module may allow a user to implement electrostimulation using the electrodes 106 to begin inducing a sweat flow. The electrostimulation may trigger sweat stimulating agents which may trigger the flow of sweat. The iontophoresis control module may also allow a user to implement release of a hydrogel agent to continue to induce a sweat flow. The iontophoresis control module may also allow a user to set a duration for the collection of refreshed sweat samples.

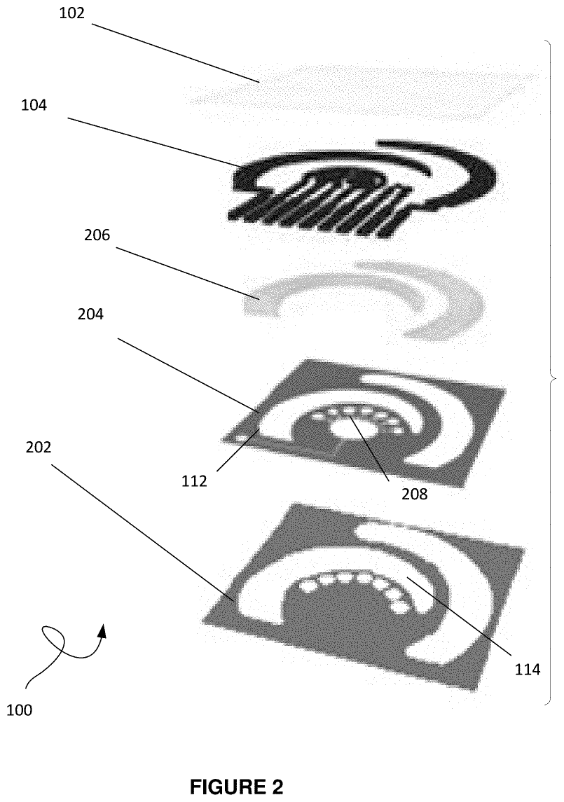

Referring now to FIG. 2 is an exploded view of the sweat sensor patch 100. The sweat sensor patch 100 may include a backing layer 102. The backing layer 102 may be made of a polyimide film. The sensor patch 100 may also include a layer having a biosensor array 104. The biosensor array 104 may be printed onto the backing layer 102 using LEG technology. The sensor patch 100 may also include a hydrogel layer 206. The hydrogel layer 206 may include a hydrogel agent applied in the same area as the electrodes 106. The hydrogel agent may be a carbachol gel (carbagel). The sensor patch 100 may also include a channel layer 204. The channel layer may include inlets 114, an outlet 112, and a reservoir 208. The sensor patch 100 may also include an inlet layer 202. The inlet layer may include inlets 114.

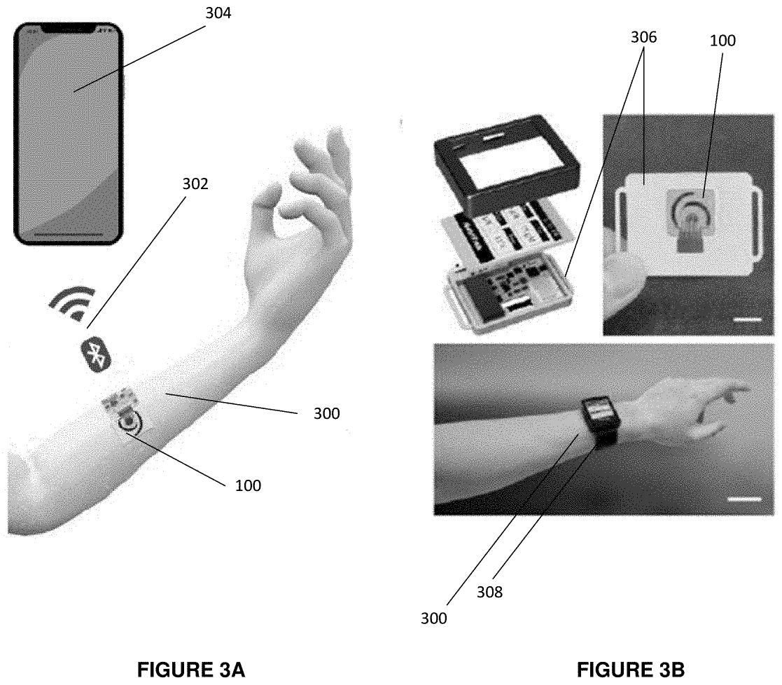

Referring now to FIG. 3A, an example of a sweat sensor system is shown. The sweat sensor system may include a sweat sensor patch 100. The sweat sensor patch may applied to a skin area 300 of a human patient. The sweat sensor patch 100 may be configured for wireless communication 302 with a mobile device 304. Wireless communication may occur via Wi-Fi or via Bluetooth. The mobile device 304 may be equipped with an application. The application may display detected health information from the sensor patch 100. The application may also be used to analyze and/or organize collected health data from the sensor patch 100.

Referring now to FIG. 3B, another example of a sweat sensor system is shown. The sweat sensor system may include a sweat sensor patch 100. The sweat sensor patch may be situated on a sweat sensor patch layer 306. The sweat sensor patch layer may be integrated into a smartwatch device 308. The smartwatch device 308 may be worn by a human patient such that the sweat sensor patch 100 contacts a skin area 300 of the human patient. The smartwatch may communicate directly with the sweat sensor system through a wired interface. The smartwatch may also communicate with the sweat sensor system through wireless communication, including over Wi-Fi and Bluetooth. The smartwatch device may display health information collected form the sweat sensor patch 100. The smartwatch device may also be used to analyze and/or organize collected health data from the sweat sensor patch 100.

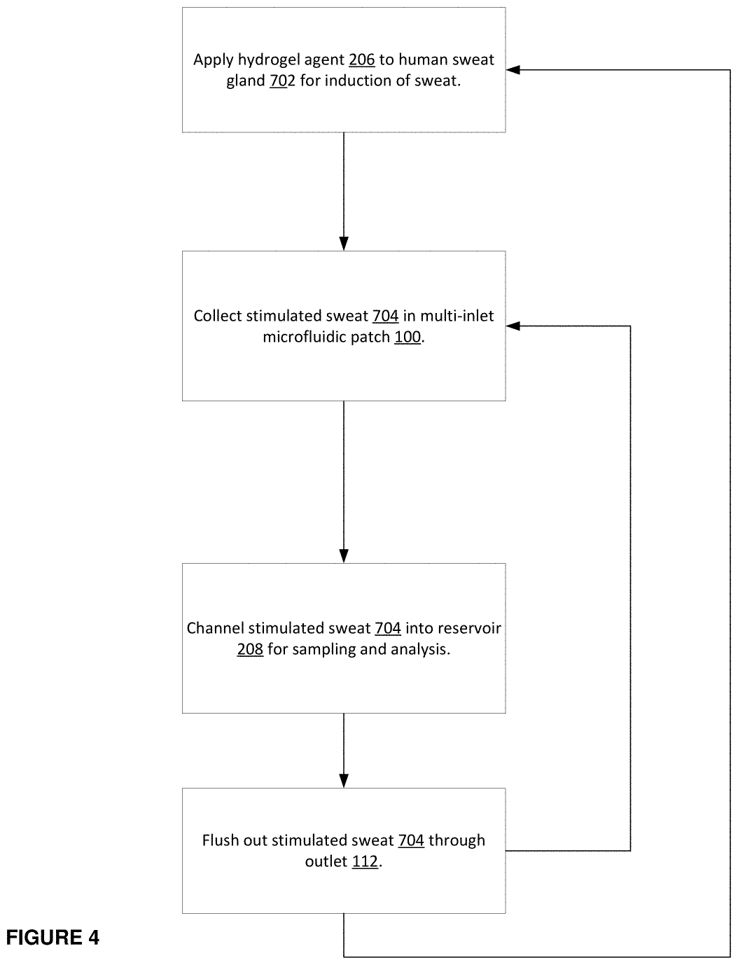

Referring now to FIG. 4, an example of a flow diagram showing a method for sweat induction and collection is shown. First, a hydrogel agent 206 is applied to a human sweat gland 702 to induce a flow of sweat. The hydrogel agent 206 may be carbagel. Next, the stimulated sweat 704 is collected in a multi-inlet microfluidic sweat sensor patch 100. The induced sweat flows in through one of the inlets 114. Next, the induced sweat sample 704 is channeled from an inlet 114 to the reservoir 208. Once in the reservoir 208, the sweat sample can be analyzed. After the sweat sample 704 is analyzed, the sweat sample 704 can be flushed out through an outlet 112. The reservoir 708 is now ready to accept a new, refreshed sweat sample. The hydrogel agent 206 may support a continuous flow of sweat over a period of time. A refreshed sample can be collected without re-application of a hydrogel agent 206 for a period of time. A period of time may be from about two hours up to a full, twenty-four hour day. Refreshed samples may be continuously collected in the multi-inlet microfluidic patch 100, channeled into the reservoir 208, analyzed, and then flushed out through the outlet 112. The entire process illustrated above is merged and integrated on a single sweat sensor patch 100. After a full day, a new sweat sensor patch 100 with new hydrogel 206 may be applied and the process shown in FIG. 4 may be repeated. The process may be repeated on a daily basis for an extended period of several days, weeks, or even months. The process may also be resumed after a break of a period of days, weeks, or months, to evaluate a change in a medical condition.

A sweat sensor patch and sweat sensor system, as described in reference to FIGS. 1-3, above, may measure concentrations of many different molecules and/or organic compounds. In one embodiment, a sweat sensor system may measure the concentrations of all or any of the nine essential amino acids. Amino acids are organic compounds that are present in the human body and in food sources. Concentrations of amino acids may vary depending on many factors including dietary intake, genetic predisposition, gut microbiota, environmental factors, lifestyle factors including sleep and exercise, and other factors. The concentrations of amino acids present in human bodily fluids, including sweat and blood, can provide important information about the health of an individual. For example, elevated levels of branched-chain amino acids (BCAAs) including for example, leucine (Leu), isoleucine (Ile), and Valine (Val) may be correlated with certain health conditions including obesity, insulin resistance, diabetes, cardiovascular disease, and pancreatic cancer. Deficiencies in amino acids, including, for example, arginine and cysteine, may indicate immune suppression and/or reduced immune-cell activation

In another embodiment, a sweat sensor may measure concentrations of amino acids in addition to other organic compounds, including vitamins and minerals. For example, imbalances with Tryptophan (Trp), tyrosine (Tyr) and phenylalanine (Phe), which are needed to support neurotransmitters such as serotonin, dopamine, norepinephrine, and epinephrine, may indicate neurological and/or mental health conditions. Other metabolic indicators involving, for example, Leu, Phe, and vitamin D, may be linked with severity, vulnerability, and mortality related to viral infections including COVID-19. Other compounds, like glucose and uric acid may also be measure to determine risk of developing and/or severity of a particular health condition.

In another embodiment, amino acids, vitamins, and mineral concentrations may be measured to develop a personalized nutrition plan. After measurement of initial concentrations, a human patient may be advised to make dietary modifications to account for deficiencies and/or excesses of key amino acids, vitamins, and minerals. The human patients adherence to a nutritional plan and progress may be monitored continuously with the sweat sensor patch.

In another embodiment, stress and fatigue detection and evaluation may be made based on concentrations of relevant metabolites. An object model for stress and fatigue may be trained. For example, the object model may be trained with standard stress and fatigue questionnaires. Then, machine learning methods may be used to optimize detection and evaluation of stress and fatigue through metabolic analysis, using questionnaires as an object model. For example, a machine learning model may optimize which metabolites are most accurately correlated with stress and fatigue determinations. A machine learning model may further optimize the level of detected metabolites which correlate most accurately to noteworthy stress and fatigue related health conditions. A machine learning model may be leveraged to determine at which point a human patient is experiencing too much stress and fatigue to be effective in a given role.

In another embodiment a sweat sensor may detect and measure drug compounds present in the sweat sample. Drug compounds may be measured to assess compliance with a drug treatment regimen. Drug compounds may also be measured to assess successful metabolization of a treatment drug. Drug compounds may also be measured to determine the risk and/or severity of drug toxicity due to a drug treatment regimen.

In another embodiment, the sweat sensor patch may measure the concentration of certain hormones. In another embodiment, the sweat sensor patch may measure the concentration of antibodies present in a human patient which may indicate an infection, the degree of immune response to a viral, bacterial, or fungal agent, an autoimmune disease, or another health condition.

A sweat sensor patch 100 may employ various power sources. For example, in one embodiment, a sweat sensor patch may be equipped with a lightweight battery. In another embodiment, the sweat sensor patch may leverage a biofluid powering system to power the device with the collected sweat flow itself. In another embodiment, the sweat sensor patch may be powered with a small solar panel. In another embodiment, the sweat sensor patch may be powered by human motion.

Polymer Detection

A MIP organic compound detection module may optimize polymer detection by creating a binding site layer in an LEG-MIP electrode. Preferred monomers may be identified for target molecules which are desirable to measure. In an embodiment, the module may use machine learning to optimize polymer detection. For example, machine learning methods may include gradient-boosted decision trees, neural networks, support vector machines, and other types of machine learning methods. Machine learning techniques may involve training an object model with template molecules to perform optimal distinction between template molecules. Selection of parameters including the monomer, crosslinker, template ratio, incubation period, and other factors may be optimized in the MIP module to achieve sensitive and selective detection of analytes. In an embodiment, polymer detection may be accomplished using a selective binding MIP layer on the LEG. MIPs may be chemically synthesized biomimetic receptors.

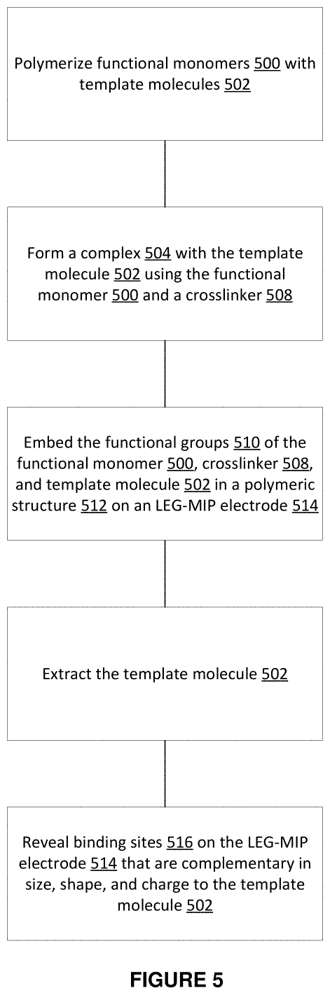

Referring now to FIG. 5, a flow diagram showing an example of a polymer detection method is shown. First, functional monomers 500 may be polymerized with template molecules 502. A preferred functional monomer 500 may be identified for each target molecule. A template molecule 502 may be a template for a target molecule which is desirable to detect. For example, in an embodiment measuring concentrations of amino acids which may indicate the presence of metabolic syndrome, measurement of the concentration of leucine may be desirable. In that case, the template molecule may be a template for leucine.

Next, a complex may be formed using the template molecule 502, monomer 500, and crosslinker 508. The functional monomer may be, for example, pyrrole. The crosslinker may be, for example, 3-Aminophenylboronic acid. Then, after polymerization, the functional groups of the functional monomer 500, crosslinker 508, and template molecule 502 may be embedded into the polymeric structure 512 on a LEG electrode 514. Next, the template molecule 502 may extracted. Extracting the template molecule 502 may reveal a binding site in the LEG electrode 514 that is complementary in size, shape, and charge to the template molecule 502. The LEG electrode 514 may, through this process, become a LEG-MIP electrode equipped to detect the desired target molecule corresponding to the template molecule. The detection may be accomplished without washing steps.

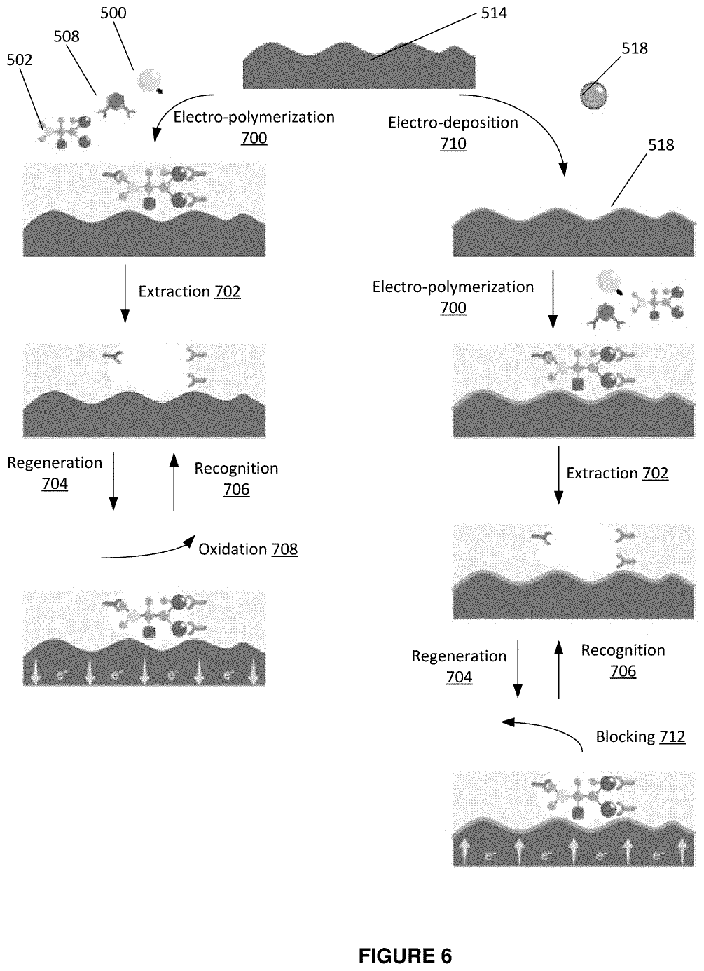

Referring now to FIG. 6, a diagram showing examples of indirect and direct detection methods for a target molecule are shown. In one embodiment, a target molecule may be detected directly after a certain period of incubation. In other words, the oxidation of the target molecule may be measured directly by differential pulse voltammetry (DPV). In DPV, a peak height correlates with an analyte concentration. A direct detection approach may be effective for electroactive molecules. However, different electroactive molecule may be oxidized at similar, and difficult to distinguish, potentials. The approach shown in FIG. 6 is still sufficiently selective and sensitive to distinguish between molecules. Several factors may influence the sensitivity including the selected monomer, the selected crosslinker, template ratios, incubation periods, and other factors. An object model may be trained with template molecules for optimized distinction between two template molecules having similar oxidization peaks. Machine learning techniques may then be used to optimize the selection of monomer, crosslinker, template ratio, incubation period, and other factors.

For the direct detection approach, the first step may be electro-polymerization of a monomer 500, crosslinker 508, and template molecule 502. The next step may be extraction 702 of the template molecule 502. The next step may be a cycle of regeneration 704 and recognition 706 of the template molecule 502. The final step may be oxidation 708, at which point the oxidation peak of a target molecule may be measured to determine the concentration of the target molecule.

In another embodiment, a target molecule may be detected indirectly. An indirect detection method may include deposition of a redox-active nanoreporter (RAR) layer between LEG and MIP layers. The RAR layer may comprise, for example, Prussian blue nanoparticles. The RAR layer may enable rapid quantification. Target molecules may then be selectively absorbed into the MIP layer which may decrease exposure of the RAR layer to the sample. In such an instance, a RAR layer may experience a diminished oxidation peak in the presence of a selectively absorbed target molecule. Therefore, using a DPV technique, as above, the RAR oxidation peak height decrease (instead of increase in the direct measurement case) may correspond to a target molecule. An indirect approach may be effective for detecting the levels of non-electroactive metabolites.

Referring again to FIG. 6, an indirect detection method may first include electro-deposition 710 of a RAR layer 518 onto an LEG electrode 514. The next step may be electro-polymerization of a monomer 500, crosslinker 508, and template molecule 502. The next step may be extraction 702 of the template molecule 502. The next step may be a cycle of regeneration 704 and recognition 706 of the template molecule 502. The final step may be a blocking 714 of the template molecule 502, in which the decrease in oxidation peak measure at the RAR layer may correspond to the concentration of the target molecule. In an embodiment, indirect LEG-RAR-MIP sensors may be regenerated in situ, upon constant potential applied to a working electrode, or other electrochemical techniques (e.g. cyclic voltammetry) in other embodiment. The applied potential may repeal bound target molecules form the MIP layer which may prolong re-usability.

In an embodiment of direct LEG-MIP sensing, discussed above, a DPV scan in sweat may occur even before target molecule recognition which may lead to an oxidation peak as a small amount of electroactive molecules. This may be oxidized on the surface of the MIP layer. After recognition and binding of the target molecule into the MIP cavities, a substantially higher current peak may be obtained. Measuring the difference in height between the initial oxidation peak and the higher peak may allow for more accurate bound target molecule measurement directly in a biofluid with high selectivity. Further, the influence of temperature and ionic strength on the metabolite sensor may be calibrated in real time based on readings from the LEG-based temperature sensor and electrolyte sensor. Calibrating these measurements may allow for continuous, accurate readings on the body during use of the sensor.

### Optimization of Microfluidic Sweat Collection Patch

A microfluidic sweat collection patch may be optimized to achieve the most rapid refreshing time between samples. Several parameters may be selected for optimization. These parameters may include, for example, the placement of inlets relative to each other and a reservoir, the number of inlets, the orientation of the inlet channels, the distance between the inlets, the distance between each inlet and the reservoir, and other factors.

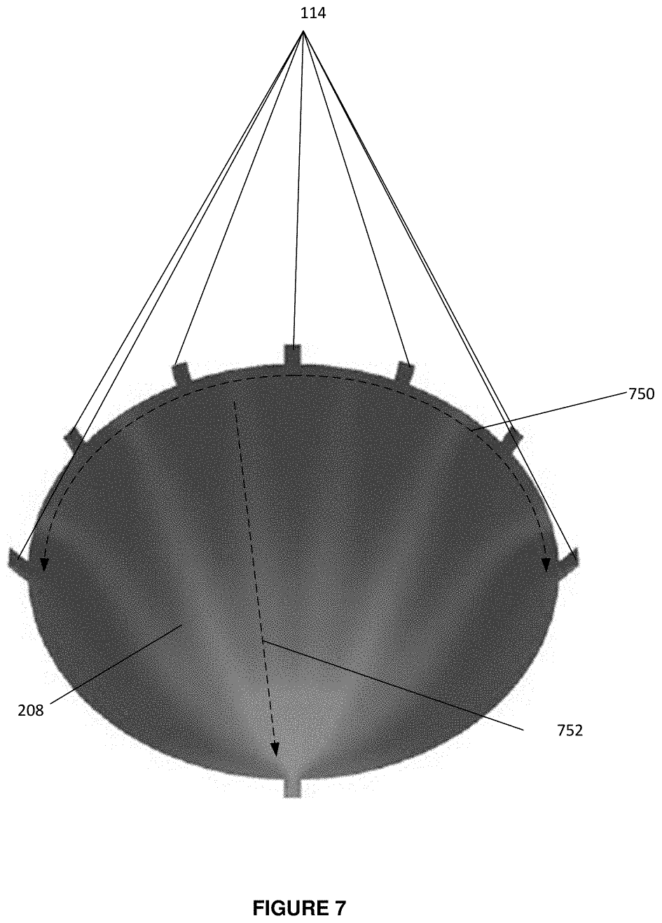

Referring now to FIG. 7, an example diagram of a microfluidic sweat collection patch is shown. The patch may include a plurality of inlets 114. The patch may also include a reservoir 208. The inlets may be configured relative to each and the reservoir at a selected angular span 750. The inlets may also be positioned to have a selected flow direction 752 relative to the reservoir 208. As shown, for example, in FIG. 7 the number of inlets may be seven. In an embodiment, the number of inlets may range from about one to ten inlets. The inlets may be positioned with an angular span of 180 degrees. The inlets may be positioned to have a flow directed toward the outlet.

A microfluidic sweat collection patch may also be designed to eliminate leakage of a sweat sample. For example, the electrostimulation may be applied to several neighboring sweat glands while avoiding the sweat glands directly underneath inlets. The patch may be designed to allow for collection of a sweat sample from only glands not in touch with the hydrogels and prevent leakage of sweat from the neighboring sweat glands (which mixed with hydrogel). This may be achieved through application of pressure on the gland the sample is taken from and through application of specialized adhesive taping of the neighboring glands and use of secure adhesive to attach the skin patch. The application of hydrogel may also be limited to optimal parts of the patch to minimize interference.

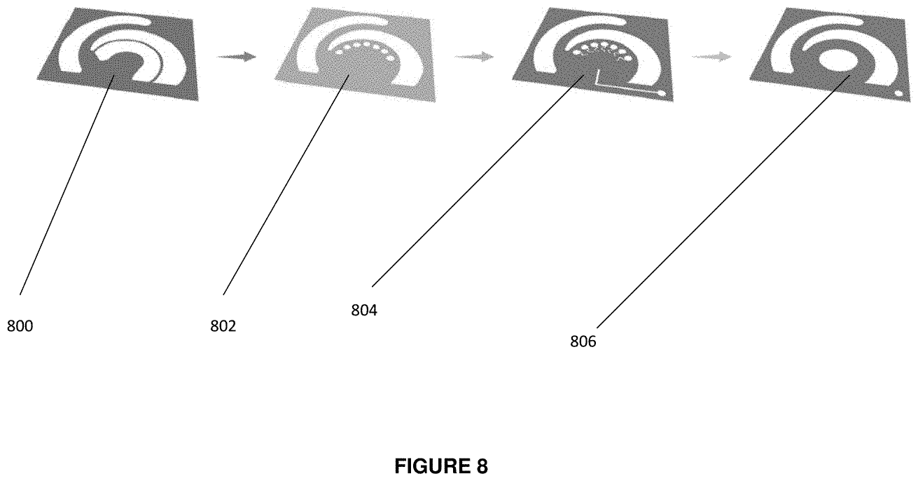

Referring now to FIG. 8, an example diagram of a microfluidic sweat collection and sampling module is shown. The module may include layers of double-sided and single-sided medical adhesives. The module may include a polyimide electrode layer. The layers of adhesives may be patterned with channels, inlets, hydrogel outlines, and reservoirs. Hydrogel outlines may be patterned to enable a flow of sweat from the top of the polyimide electrode layer. The module may include a bottom layer 800 which may be a double-sided adhesive layer in direct contact with a skin area. This bottom layer may be patterned with an accumulation well to collect sweat. The module may also include an inlet layer 802 in direct contact with the bottom accumulation layer. The inlet layer may contain a plurality of sweat inlets. The module may also include a channel layer 804 patterned with a plurality of microfluidic channels. The channel layer may be in direct contact with the inlet layer. Sweat collected in accumulation wells may flow to the inlets and then in turn flow through the channels. The module may also include a reservoir layer 806 which may be patterned with a reservoir and an outlet. Sweat may flow through the channels into the reservoir. After sampling, sweat may flow out through the outlet. The reservoir layer may lie between the channel layer and the polyimide electrode layer.

While various embodiments of the present invention have been described above, it should be understood that they have been presented by way of example only, and not of limitation. Likewise, the various diagrams may depict an example architectural or other configuration for the invention, which is done to aid in understanding the features and functionality that can be included in the invention. The invention is not restricted to the illustrated example architectures or configurations, but the desired features can be implemented using a variety of alternative architectures and configurations. Indeed, it will be apparent to one of skill in the art how alternative functional, logical or physical partitioning and configurations can be implemented to implement the desired features of the present invention. Also, a multitude of different constituent module names other than those depicted herein can be applied to the various partitions. Additionally, with regard to flow diagrams, operational descriptions and method claims, the order in which the steps are presented herein shall not mandate that various embodiments be implemented to perform the recited functionality in the same order unless the context dictates otherwise.

Although the invention is described above in terms of various exemplary embodiments and implementations, it should be understood that the various features, aspects and functionality described in one or more of the individual embodiments are not limited in their applicability to the particular embodiment with which they are described, but instead can be applied, alone or in various combinations, to one or more of the other embodiments of the invention, whether or not such embodiments are described and whether or not such features are presented as being a part of a described embodiment. Thus, the breadth and scope of the present invention should not be limited by any of the above-described exemplary embodiments.

Terms and phrases used in this document, and variations thereof, unless otherwise expressly stated, should be construed as open ended as opposed to limiting. As examples of the foregoing: the term “including” should be read as meaning “including, without limitation” or the like; the term “example” is used to provide exemplary instances of the item in discussion, not an exhaustive or limiting list thereof; the terms “a” or “an” should be read as meaning “at least one,” “one or more” or the like; and adjectives such as “conventional,” “traditional,” “normal,” “standard,” “known” and terms of similar meaning should not be construed as limiting the item described to a given time period or to an item available as of a given time, but instead should be read to encompass conventional, traditional, normal, or standard technologies that may be available or known now or at any time in the future. Likewise, where this document refers to technologies that would be apparent or known to one of ordinary skill in the art, such technologies encompass those apparent or known to the skilled artisan now or at any time in the future.

The presence of broadening words and phrases such as “one or more,” “at least,” “but not limited to” or other like phrases in some instances shall not be read to mean that the narrower case is intended or required in instances where such broadening phrases may be absent. The use of the term “module” does not imply that the components or functionality described or claimed as part of the module are all configured in a common package. Indeed, any or all of the various components of a module, whether control logic or other components, can be combined in a single package or separately maintained and can further be distributed in multiple groupings or packages or across multiple locations.

Additionally, the various embodiments set forth herein are described in terms of exemplary block diagrams, flow charts and other illustrations. As will become apparent to one of ordinary skill in the art after reading this document, the illustrated embodiments and their various alternatives can be implemented without confinement to the illustrated examples. For example, block diagrams and their accompanying description should not be construed as mandating a particular architecture or configuration.

The terms “substantially,” “approximately,” and “about” are used throughout this disclosure, including the claims, are used to describe and account for small fluctuations, such as due to variations in processing. For example, they can refer to less than or equal to ±5%, such as less than or equal to ±2%, such as less than or equal to ±1%, such as less than or equal to ±0.5%, such as less than or equal to ±0.2%, such as less than or equal to ±0.1%, such as less than or equal to ±0.05%.

What is claimed is:

1. A biosensor patch comprising:
an iontophoresis module configured to stimulate production of a biofluid sample;
a microfluidic collection and sampling module configured to collect and sample the biofluid sample stimulated by the iontophoresis module; and
an electrochemical analyte sensor module configured with recognition elements to bind to and detect target molecules present in the biofluid sample stimulated by iontophoresis module and collected in the microfluidic collection and sampling module.

2. The biosensor patch of claim 1 wherein the analyte detection module comprises a molecularly imprinted polymer (MIP) organic compound sensor module, wherein the MIP is imprinted to match binding sites of target molecule to detect target molecules present in the biofluid sample collected in the multi-inlet microfluidic collection and sampling module.

3. The biosensor patch of claim 2 wherein the MIP organic compound sensor module is configured to detect a target molecule by:
regenerating the target molecule;
recognizing of the target molecule;
oxidizing of the target molecule; and
detecting the concentration of the target molecule based directly on the measured oxidation peak of the target molecule.

4. The biosensor patch of claim 2 wherein the MIP organic compound sensor module is configured to detect the target molecule by:
performing electro-deposition of a redox-active nanoreporter (“RAW”) layer onto the LEG;
regenerating the target molecule;
recognizing the target molecule;
measuring a decrease in oxidation peak at the RAR layer; and
detecting the concentration of the target molecule based indirectly on the measured decreased oxidation peak.

5. The biosensor patch of claim 1 wherein the microfluidic collection and sampling module comprises:
inlets, each inlet providing a channel for the inflow of a biofluid sample; and
a reservoir connected to the inlets such that refreshed biofluid samples accumulate in the reservoir; and
an outlet providing a channel for the outflow of the biofluid sample.

6. The biosensor patch of claim 5 comprising a multi-inlet configuration wherein the inlets are positioned relative to the reservoir at a selected angular span and wherein the inlet channels follow a selected orientation relative to the reservoir

7. The biosensor patch of claim 6 wherein the microfluidic collection and sampling module comprises seven inlets and wherein:
the selected angular span of the inlets is about 180 degrees; and
the selected orientation requires that the inlet channels are aligned toward the outlet.

8. The biosensor patch of claim 1 wherein the patch further comprises:
an accumulation layer having accumulation wells and adhesive, wherein the accumulation layer is directly affixed to a skin area with the adhesive and wherein biofluid accumulating on the skin surface is collected in the accumulation wells;
an inlet layer directly affixed to the accumulation layer, the inlet layer having inlets wherein biofluid flows from the accumulation wells into the inlets;
a reservoir layer directly affixed to the inlet layer, the reservoir layer having a reservoir and an outlet and wherein biofluid flows from the channels into the reservoir and, after sampling of the biofluid, the biofluid exits through the outlet; and
a flexible plastic electrode layer directly affixed to the reservoir layer configured with an outlet providing for the exit of the biofluid.

9. The biosensor patch of claim 8 further comprising a channel layer directly affixed to the inlet layer, the channel layer having a plurality of channels wherein biofluid flows from the inlets into the channels.

10. The biosensor patch of claim 1 wherein the analyte sensor module is fabricated using laser-engraved graphene (“LEG”) technology.

11. The biosensor patch of claim 1 further comprising an in situ signal processing and wireless communication module.

12. The biosensor patch of claim 1 further comprising adhesive backing for direct application to skin.

13. A method for configuring a MIP to detect a target molecule comprising:
polymerizing functional monomers with template molecules;
forming a complex with the target molecule using the functional monomer and a crosslinker;
embedding the functional groups of the functional monomer and crosslinker in a polymeric structure on laser engraved graphene (“LEG”);
extracting the target molecule;
revealing binding sites on the LEG-MIP electrode that are complementary in size, shape, and charge to the target molecule.

14. A continuous health monitoring system comprising:
a biofluid induction agent, wherein the biofluid induction agent stimulates production of biofluid;
a biofluid sampling and collection module, wherein the module collects the induced biofluid sample for analysis;
a metabolite detection module, wherein the metabolite detection module identifies concentrations of target metabolites present in the collected biofluid sample; and
a smart device, wherein the smart device displays collected health information.

15. The system of claim 14 wherein all components are fully integrated into a wearable smart watch device.

16. The system of claim 14 wherein the mobile device is equipped with a mobile application for displaying, processing, and storing collected health data.

17. The health monitoring system of claim 14 wherein target metabolites comprise amino acids.

18. The health monitoring system of claim 14 wherein target metabolites comprise hormones.

19. The health monitoring system of claim 14 wherein a target metabolite comprises glucose.

20. The health monitoring system of claim 14 wherein a target metabolite comprises uric acid.
